(19) United States
(12) Patent Application Publication
(10) Pub. No.: US 2013/0004544 A1
(43) Pub. Date: Jan. 3, 2013

(54) Synthetic Antibacterial Clay Compositions and Method of Using Same

(76) Inventors: David W. Metge, Lafayette, CO (US); Lynda Williams, Tempe, AZ (US); Dennis D. Eberl, Boulder, CO (US); Alex E. Blum, Boulder, CO (US); Ronald W. Harvey, Boulder, CO (US)

(21) Appl. No.: 13/515,309

(22) PCT Filed: Jun. 1, 2010
(86) PCT No.: PCT/US2010/001591

§ 371 Date: Sep. 10, 2012

Related U.S. Application Data
(60) Provisional application No. 61/183,451, filed on Jun. 2, 2009.

(51) Int. Cl. A61K 33/26; C12Q 1/18; A61P 31/04; A61K 9/14

(52) U.S. Cl. 424/400; 424/646; 435/32

(57) ABSTRACT

The present invention is directed to synthetic bactericidal compositions having clay like properties and a method of using these compositions to topically treat bactenally-caused skin infections and skin diseases. The compositions within the scope of the invention compnse a bactericidal effective amount of a reducing agent, such as pyπte, marcasite, pyrrhotite, FeS2, FeS, FeS04—or other reducing agents having like properties, and a natural clay or clay mineral and/or synthetic clay or clay mineral, or other suitable matenals having clay-like properties. The synthetic bactericidal compositions are synthesized by adding the reducing agent to the clay or clay mineral. It is the presence of the reducing agent in said compositions that renders them bacteπcidal. The clays serve as a vehicle within which the reducing agent is dispersed, as a diluent to the reducing agent, and also as an adsorbent and low permeability banner in use of the composition

### GOVERNMENT INTEREST

The invention described herein may be manufactured, used, and licensed by or for the United States Government.

### FIELD OF INVENTION

The present invention relates to synthetic antibacterial compositions having clay-like properties and a method of using these compositions to topically treat bacterially-caused skin infections and skin diseases. The compositions within the scope of the invention comprise a bactericidal effective amount of a reducing agent, such as pyrite, marcasite, pyrrhotite, FeS₂, FeS, FeSO₄, or other reducing agents having like properties, and a natural clay or clay mineral and/or a synthetic clay or clay mineral, or other suitable materials having clay-like properties. Divalent iron within the structure of a clay mineral itself may also serve as a reducing agent. It is the presence of the reducing agent in said compositions that renders them antibacterial. The clays or clay minerals serve as a vehicle within which the reducing agent is dispersed, as a dilutent to the reducing agent, and also as an adsorbent and low permeability barrier in use of the composition. These compositions are hydrated so as to form a paste, which is then to be applied to the affected area for treatment. Compositions within the scope of the present invention are suitable to topically treat infections and skin diseases caused by one or more types of bacteria, including antibiotic-resistant bacteria.

### BACKGROUND OF THE INVENTION

Antibiotics are available to treat various types of skin infections. Their route of administration for such treatments can vary. Some are prescribed to be taken orally, some to be applied topically, while others to be administered intravenously. The present invention relates to an antibacterial composition that is to be applied topically to an affected area. In particular, the present invention relates to synthetic antibacterial clay (ABC) compositions.

The term “antibacterial” is used in the title herein, because an antibacterial clay or clay mineral may either kill bacteria, and therefore be “bactericidal,” or render them bacteriostatic, which means that the bacteria cannot grow or reproduce. However, the staining technique used to distinguish between viable and non-viable bacteria herein indicates that most of the bacteria have been killed in the experiments to be described herein, and, therefore, the terms antibacterial and bactericidal are used interchangeably herein. Many agents that are bactericidal also are antimicrobial, which means that the antimicrobial agents may attack bacteria, viruses, protozoa, fungus, etc. Applicants have not yet tested the antibacterial clays against other types of microbes, but speculate that these clays may be effective against some of these entities as well.

Studies have been conducted exploring the potential use of natural clays in the topical treatment of bacterial infections. For example, certain natural clays have been used in the Ivory Coast to treat Buruli ulcer: see http://fifthkingdom.net/BuruliBusters/default.htm. Although researchers have identified a few natural clays as having bactericidal activity, these studies have not been conclusive as to how or why these clays are effective. Additionally, these studies did not provide definitive guidance as to how one having ordinary skill in the art would conclusively be able to select or identify suitable antibacterial natural clays, or, for that matter, synthesize suitable antibacterial clay-like or clay containing compositions for topical treatment of bacterial infections. Applicants are not aware of any specific guidance present in the art that might direct one skilled in the art to do so. Mechanistically, it has been unclear how these clays produce bactericidal activity.

According to Williams et al., [Williams et al., “Chemical and Mineralogical Characteristics of French Green Clays Used for Healing,” Clays and Clay Minerals, Vol. 56, No. 4, 437-452 (2008)], the current accepted treatment of M. ulcerans ulcers larger than 5 cm is surgical excision, limb amputation, and/or subsequent skin grafting.

Certain natural clays have been used in the Ivory Coast to treat Buruli ulcer, a flesh-eating disease caused by Mycobacterium ulcerans. This has been documented in O'Hanlon, “Medicinal Clays May Heal Ulcers,” News in Science, 26 Oct. 2007. This article reports a French clay identified as Agricur* as effective against this flesh-eating disease in Africa's Ivory Coast, and that an interdisciplinary team of microbiologists and mineralogists was attempting to discover how the clay cures. (*Per applicants, although the article references this clay as “Agricur,” this clay is correctly referred to as “Argicur,” carrying the company/supplier name, Argicur Inc. Le Buisson de Cadouin, France.) The article further indicates that two mineralogically-similar clays had different antibacterial activity. Therefore, based on this article, it appears uncertain which specific clay compositions would be effective for the treatment of bacterial infections. Research was initiated into determining what makes one clay toxic to bacteria and another harmless. The article further indicates that several well-known, pathogenic bacteria—Salmonella typhimurium, Streptococcus sp., Escherichia coli and Pseudomonas sp. —were exposed to the clays; bacterial cultures lost 90-99% (1-2 log unit loss) viability within 24 hours of exposure to French Argicur clays. This was in contrast to only 10-40% reduced viability (0.2-1 log unit loss) caused by other clays or sterile sand. Based on the article, the mechanism as to how the clay works is uncertain. It is also uncertain, based on this article, as to what would make a specific clay more suitable for such treatment, and what would motivate one having ordinary skill in the art to select one clay over another for such treatment.

Consistent with this article are the results and conclusions set forth in Williams et al., “Killer Clays! Natural antibacterial clay minerals,” Mineralogical Society Bulletin, 139: 3-8 (2004). The article references and explores the effect on bacteria of two French clays from two different suppliers. The authors reference therein that “[i]t was immediately apparent that one of the clay samples was not effective in killing Mycobacterium, but was more suited to promotion of skin granulation after the Mycobacterium were killed. These observations remain unexplained.” Further, based on the teachings therein, it appeared that one of the clays (Argiletz) apparently kills Mycobacterium ulcerans (in human trials); but, in vitro, this same clay enhanced E. coli, whereas the Argicur clay killed E. coli. Based on the article, much needs to be explored to determine what properties of clay, if any, may render it antibacterial. The authors of the article indicate that they are exploring numerous variables, such as, trace element exchange, surface free energy potential, pH, oxidation state, and how these vary with mineral morphology. No specific information is identified as to what properties and/or composition a clay must have in order to exhibit antibacterial (bactericidal) properties.

Haydel et al., “Broad-spectrum in vitro antibacterial activities of clay minerals against antibiotic-susceptible and antibiotic-resistant bacterial pathogens,” J. of Antimicrobial Chemotherapy (2008) 61, 353-361, reported that two iron-rich clay minerals, which are similar in major phases and bulk chemistry, have striking and opposite effects on bacterial populations, ranging from enhanced microbial growth to complete growth inhibition. The article states that “of the six independent clay samples collected from the French supplier and tested against various bacteria . . . only CsAg02 displayed antibacterial effects . . . there is not a single component of CsAg02 clay (e.g. transition metals) that stands out as an obvious antibacterial agent, so it may be a fortuitous combination of factors (multiple components) responsible for the inhibitory property.” (at p. 359)

Applicants studied many different natural clays and clay minerals, and the present invention is a result of these studies. For example, Applicants studied the original sample of Argicur used in the Ivory Coast study, and found it to be bactericidal. They further studied subsequent samples of the Argicur green clay collected from the company mill (Argicur Inc. Le Buisson de Cadouin, France), and found that these were not bactericidal. Therefore, although teachings exist that set forth that Argicur clays have antibacterial/bactericidal properties, it is the Applicants' experience, based on their analysis, that not all Argicur clays have similar bactericidal activity; they were not alike. In addition, of note is that most clays and clay minerals that Applicants studied were found to either have minimal to no bactericidal effect or to actually promote bacterial growth.

Through their extensive research, applicants did, however, identify a property which renders the natural antibacterial clays tested (i.e., the Argicur used in the Ivory Coast and Pyroclay) antibacterial. In particular, applicants identified that the presence of particular reducing agents (pyrite in these), in particular amounts, and in fine particle form, is what renders these natural clays antibacterial. These reducing agents were found to be absent in clays found to not have bactericidal properties. It is the identification of these reducing agents that lead to the present invention. Applicants are now able to artificially produce an antibacterial (bactericidal) composition having clay-like properties—referred to herein as synthetic antibacterial composition or synthetic bactericidal composition. These compositions may have some advantages over simply using a natural antibacterial clay in that they can be customized to illicit desired properties—i.e., purity level. In particular, the present invention relates to synthesized antibacterial compositions, containing a clay or clay mineral and a bactericidal effective amount of a reducing agent, that Applicants believe may be used to topically treat most, if not all, bacterial skin infections, including those caused by antibiotic resistant bacteria. In use, the composition within the scope of the invention is hydrated to form a paste, which is applied to the affected area. Although Brunet de Courssou reported that treatment of Buruli ulcer with clay was found to be painful, it is applicants' belief that some patients may find the treatment reasonably painless; some may even find it soothing.

### BRIEF SUMMARY OF THE INVENTION

The invention relates to synthetic antibacterial compositions for the topical treatment of bacterially-caused skin infections, and methods of using the same. These synthetic compositions have the properties of clay (clay-like properties), and contain therein an antibacterial effective amount of a reducing agent in a clay. These compositions may be prepared by adding an antibacterial effective amount of a reducing agent (for example, pyrite) to a clay or clay mineral, wherein said reducing agent renders the composition antibacterial.

The invention further relates to synthetic antibacterial clays or clay minerals, which may be prepared by synthesizing a clay or clay mineral, or by otherwise treating or altering the chemistry of a natural clay or clay mineral, to yield an antibacterial effective amount of a reducing agent within the clay mineral's crystal structure. In the latter preparation, for example, ferrous iron is incorporated into the octahedral sheet of a synthetic clay mineral, or is introduced by the reduction of ferric iron already present in the octahedral sheet. Ferrous iron also could be introduced into the exchange positions of clay minerals (for example, into the interlayer position of smectitic clay minerals) by cation exchange.

The reducing agents that the applicants have identified as suitable to render the compositions herein bactericidal are the polymorphs of FeS₂, which include pyrite and marcasite, but other similar agents may work as well, agents, such as manganese oxides, pyrrhotite, FeS, FeSO₄, and other minerals (natural or synthetic) or compounds that contain reducing transition metals with like properties, or reducing agents that are present within the structure of the clay mineral itself. Reducing agents may be employed and be present in the compositions in the form of fine (submicron) particles. Their fine particulate sizes, as well as the amounts of these reducing agents present in the composition of the invention, do not materially affect the clay-like properties of the composition.

Natural and/or synthetic clays or clay minerals are employed in the composition, and serve as a carrier for the reducing agents. They bind/sorb the reducing agent and may play a role in buffering the chemical reaction(s) produced by the reducing agent(s). They also serve as an absorbent and low permeability barrier when the composition is in use. In addition, the exchange properties of the clay mineral may enhance the solubility of sparingly soluble reducing agents, as has been found for the enhanced dissolution of other sparingly soluble compounds in the presence of montmorillonite (Eberl and Landa 1985). The reducing agent imparts antibacterial properties to the clay containing compositions herein.

Smectite-clays, illite-clays, rectorite-clays and clays having like properties, or a combination of these, may be suitably employed as the carrier for the reducing agent in the present invention. These clays may be natural or synthetic. In addition to serving as a carrier for the reducing agent, the clays serve as a low permeability barrier to keep atmospheric oxygen from reaching the skin or other surfaces being treated with the composition herein, and may serve as a scavenger for oxygen. They also must be suitable to keep the system moist. In addition to the suitability of natural and/or synthetic clays for use as the carrier herein, applicants submit that polymers, for example, and other materials having clay-like properties (for example, kaolinites, chlorites) may be employed in this role as well. One having ordinary skill in the art with knowledge of the teachings herein would be readily able to identify the types of carriers that may be suitably employed herein and within the scope of the present invention. Moreover, one having ordinary skill in the art would recognize that natural clays may require processing so as to render them suitable for use in topical pharmaceutical compositions. These treatments are well within the skill of the art, and include removal of environmental or air-borne bacteria by sterilization. Also, it may be necessary to remove accessory minerals, such as quartz, by particle size separation techniques (e.g., separation of quartz and feldspar from the clay minerals by settling in water or by centrifugation according to Stokes Law), and cation exchange to change the chemical, sorptive and swelling properties of the clay. Well known sterilization treatment may include, for example, ultraviolet (uV) or heat sterilization. These would remove, render nonlethal, or inactivate potentially pathogenic organisms such as virus, bacterial spores or protozoan cysts.

Applicants anticipate that one skilled in the art will also recognize that an antibacterial effective amount of a reducing agent may be added to any natural clay or clay mineral, regardless as to whether or not that natural clay or clay mineral has natural bactericidal properties, so as to be certain that a composition containing that natural clay is suitable for the purpose intended herein. Prior to the present invention, applicants contend that there was no motivation to add a reducing agent to a clay or clay mineral (natural or synthetic) for the use described herein.

In addition, since Applicants' research has lead them to identify that it is the presence of a reducing agent in a natural clay that is responsible for its bactericidal activity, the present invention, therefore, also relates to a method of identifying a natural clay having bactericidal activity in its natural form. This method involves chemical and mineralogical analyses of clay in determining the presence of a reducing agent therein, or measurement of the oxidation-reduction potential of a clay slurry. Once identified, these naturally-bactericidal clays can be appropriately processed to render them suitable for topical pharmaceutical use. In the past, random clays would be evaluated for their “healing” (herein, antibacterial) properties; this method of evaluation was “hit-or-miss.” With applicants' discovery as to what renders natural clays antibacterial, identifying clays having these properties has been rendered predictable. Specific motivation exists for further processing or modifying these clays to render them suitable for topical bactericidal pharmaceutical use. Modification of clays could occur, for instance, by adding a pH buffering agent such as CaCO₃ or NaHCO₃ to maintain a slightly acidic (pH 4.5-5) rather than strongly acidic (pH 2-3) environment.

Compositions within the scope of the present invention may be used to topically treat bacterial skin infections and diseases—including those caused by antibiotic-resistant bacteria, such as methicillin resistant Staph. Aureus (MRSA). Water, or other suitable pharmaceutically acceptable aqueous liquids (e.g., inert solution), are added to a composition in sufficient amounts to create a paste. It is the composition in this paste form that is topically applied to an infected area, for example, of the skin. The mechanism in which these bactericidal compositions operate is different from the mechanism of commonly used or commercially available antibiotics.

Data from Williams et al. 2008 included results for two types of French clays; they indicated that the Argiletz French green clay was not bactericidal against E. coli. By contrast, Argicur supplied green clay displayed antimicrobial properties and was found to be bactericidal against several species of bacteria, including E. coli and S. aureus. One question was whether or not changing chemical conditions mediated by clays might be important in clay bacterial activity.

Our experimentation with these clay types upon several bacterial types such as Salmonella, E. coli, Pseudomonas, Staphylococcus and Streptococcus, suggested that clay type and degree of bactericidal activity depended upon reducing conditions, possibly mediated by FeS₂ (pyrite) or by other minerals with available reduced metals. Pyrite has been implicated in spontaneous production of chemical radicals; chemical radicals such as OH. and O₂⁻, would be highly damaging to biomolecules such as sugars, fatty acids or proteins located on bacterial cell surfaces and within cells. Additionally, the Fe2+ from pyrite might produce intracellular Fenton-type reactions (described later herein). The reaction products could damage nucleic acids such as DNA or RNA or hamper cellular metabolic functions. Blue clay (a clay from Oregon that naturally contains about 10% pyrite) was determined to be bactericidal, but neither weathered Blue clay (Blue clay that had been oxidized naturally by weathering) nor Ormalite (a commercial name for a clay from a nearby deposit) were particularly bactericidal. Of note is that neither weathered Blue clay nor Ormalite were found to contain pyrite or other reducing agents.

Data from dialysis tube experiments (described later herein) indicated a clear dose-response among pyrite content, redox state and bactericidal activity. Wyoming smectite was chosen initially as an end member for montmorillonite clays since it is similar in composition to the smectite-rich Agricur clay. Whereas the Wyoming montmorillonite was only mildly bactericidal initially, the addition of pyrite (and finer grained pyrite created by grinding) increased bactericidal activity by 7- to 28-fold (against E. coli and Staphylococcus, respectively).

Two domestic clays, Pyroclay and Blue clay, were found to be bactericidal, and each contained 3 to 10% by weight pyrite. These were both somewhat reducing when mixed with water. By contrast, Blue clay in which all of the pyrite had been removed by oxidation (weathered Blue), which was mineralogically similar to antibacterial Blue clay with respect to mineral content other than the presence of pyrite (Table 1) displayed nearly 10-fold greater viable cells. This suggests that bactericidal components contained within the clay could be diminished by chemical oxidation. A clay collected from nearby the Blue Clay formation (designated with the commercial name “Ormalite”) did not contain pyrite and was not bactericidal.

It is recognized that the composition of these varied clays differ in more than pyrite content. For example, in an initial study by Williams et al, 2004 (GSA poster), studies of Pyroclay, Wyoming montmorillonite (Swy-1 from The Clay Mineral Society repository) and Argicur showed that only Argicur was antibacterial (i.e., little or no bacterial growth when the clay suspension was incubated with log-phase E. coli). By contrast, clays later confirmed as non-bactericidal (Wyoming montmorillonite and Argiletz) produced little effect upon bacterial growth when bacteria were incubated with clay suspension.

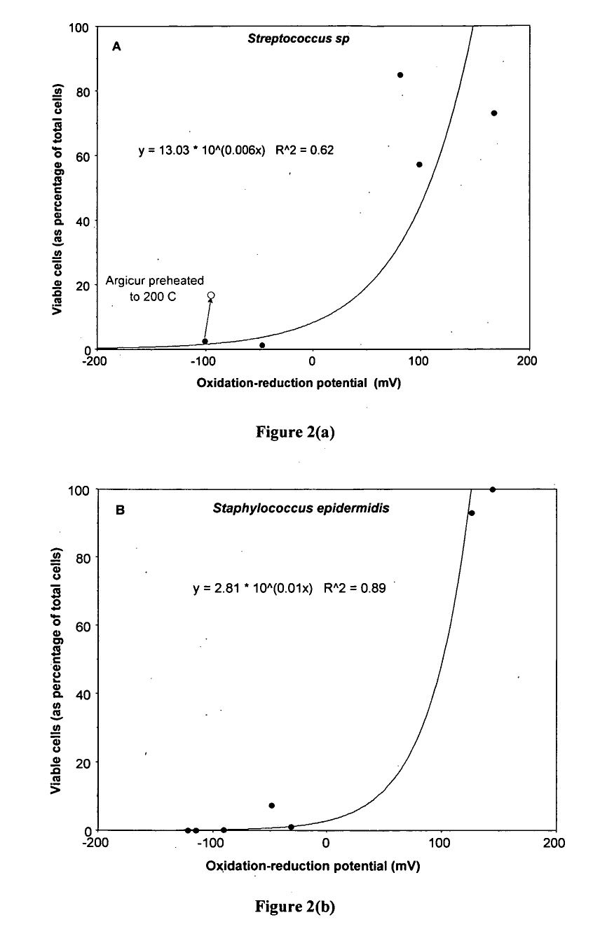

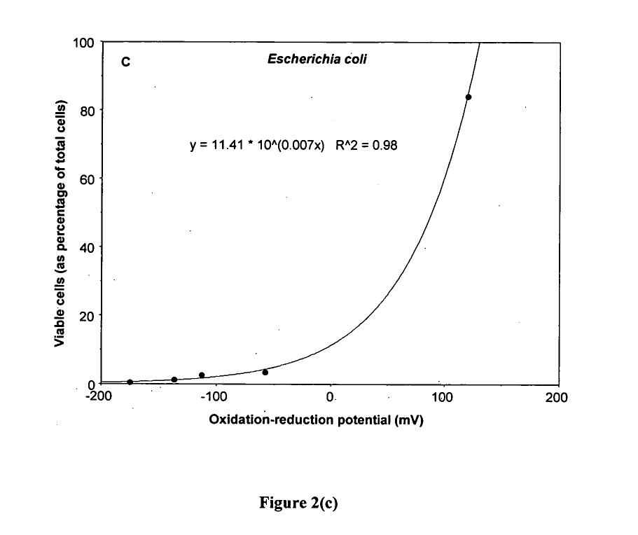

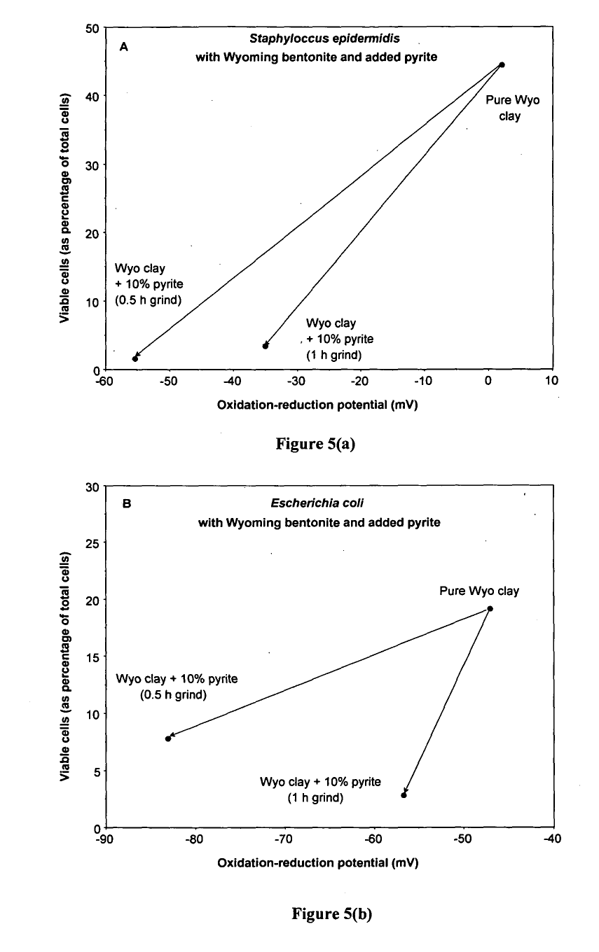

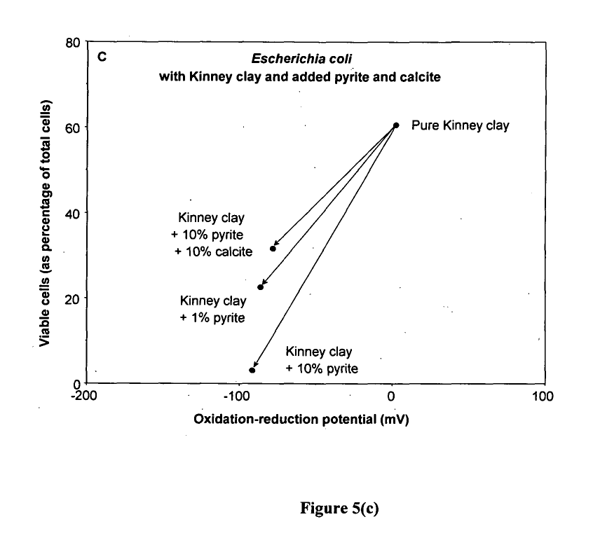

Applicants conducted experiments using Wyoming montmorillonite. These are set forth as Example 1, under Experimental Procedures and Data herein. The results are set forth in Table 4 and in FIGS. 2A, 5A and 5B.

Mediation of pH and oxidation-reduction potential (ORP) by calcite addition was tested with pyrite-containing clays. The purpose of this addition was to increase the solution pH from acidic to more circumneutral. Thus, an approximate 10% addition of calcite to the Oregon Blue clay (0.15 g calcite:1.6 g clay) produced a >3 pH unit increase (pH 2.55 to pH 5.78) in 48 hours. This was accompanied by a change in solution ORP from −44.2 mV to −20.1 mV. We would predict, based upon previous data with bactericidal and non-bactericidal clays, a slight reduction in overall bactericidal activity of the clay by less than 0.4 log unit. Thus, even with calcite mediation of approximately 10%, the bactericidal activity of this particular clay would be maintained, because about 80% of the viable bacteria would be rendered non-viable. By contrast, a 10% addition of calcite to Pyroclay raised the pH from 4.65 to 8.19 and ORP changed from −113 mV to −67.9 mV within a 48 hour period. We would predict less than a 1% change in bacterial viability out of a 2-log reduction in overall bacterial viability when calcite is not added. Finally, addition of a 10% calcite to a Kinney montmorillonite+10% pyrite mixture resulted in an alkaline pH (8.26), and a slightly more positive ORP (−78.4 mV) than the Kinney+10% pyrite amendment mixture without calcite (pH 5.66, ORP −91.9 mV). This slight difference in ORP and pH was accompanied by an order of magnitude difference in bactericidal activity (3.1% viable cells for pyrite amended Kinney clay compared to 31.7% viable cells for Kinney clay amended with calcite and pyrite), Clearly, calcite is able to both mediate pH and substantially moderate the pyrite-amended bactericidal activity for this clay type.

### DESCRIPTION OF THE TABLES

Table 1 provides applicants' previously undisclosed determination of the quantitative mineralogical composition of most of the clays used in the experiments. The table shows that these clays contain non-clay minerals in addition to clay minerals. Weathered Blue clay, Argiletz, Miraculite and Ormalite are non-antibacterial clays (NABC), whereas Argicur, Pyroclay and Blue clay are antibacterial clays (ABC). The Argicur sample listed in this table is the same as the Argicur used in the Ivory Coast studies referenced above. North Sea sediment is not presented in this table because it was analyzed qualitatively rather than quantitatively. It is estimated to contain about 5% pyrite.

Table 2 provides chemical analyses for the pure smectites used in some of the experiments. These analyses are used to calculate the structural formulae for these clay minerals, given in Table 3.

Table 3 provides structural formulae for two pure smectites (<1 micron size fractions) used in the experiments, based on the analyses given in Table 2. Both the Kinney and the Wyoming smectites are montmorillonites (that is, the negative 2:1 layer charge is located mainly in the octahedral sheet), but the Wyoming clay has both octahedral ferrous (Fe²⁺) and ferric (Fe³⁺) iron, whereas the Kinney clay contains only ferric iron. It is postulated that it is ferrous iron in the Wyoming structure that renders this montmorillonite in its pure form more antibacterial than the Kinney montmorillonite (19.1% viable E. coli cells remaining versus 60.6% for the Kinney; see Table 4).

Table 4 sets forth a comparison of bacterial viability results for three different species of bacteria versus oxidation-reduction potential for mineral slurries. These slurries were composed of 40 mg/mL untreated clays, untreated and treated clay minerals, pyrite-amended clay minerals, or pyrite. Of particular interest is the affect of the different types of clays and pyrite mineralogies upon bacterial viability. The data set forth are a summary of data obtained from dialysis tubing experiments described under Experimental Procedures and Data section herein. The data in Table 4 are used in drawing FIGS. 2, 3, 5 and 6, herein.

### DETAILED DESCRIPTION OF THE FIGURES

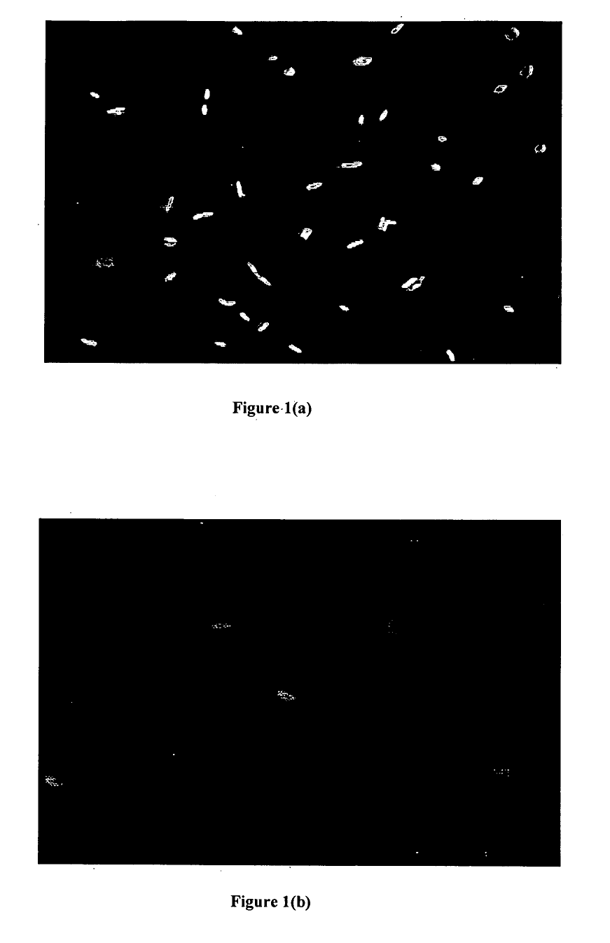

FIG. 1(a) shows a photomicrograph of E. Coli subjected to Kinney montmorrillonite leachate at 788× magnification at 24 h.

FIG. 1(b) shows a photomicrograph of E. Coli subjected to 10% pyrite leachate at 788× magnification at 24 h;

FIG. 2(a) shows the percent of viable cells at the end of dialysis tube experiments on Streptococcus sp;

FIG. 2(b) shows the percent of viable cells at the end of dialysis tube experiments on Staphylococcus epidermis;

FIG. 2(c) shows the percent of viable cells at the end of dialysis tube experiments on Escherichia coli;

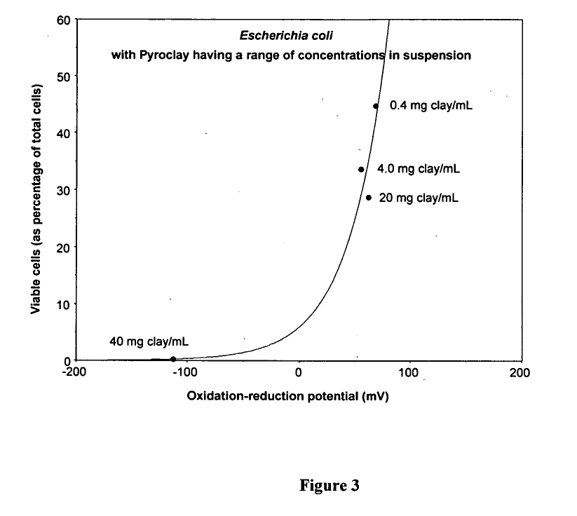

FIG. 3 shows the percentage of viable Escherichia coli cells at the end of the experimentation;

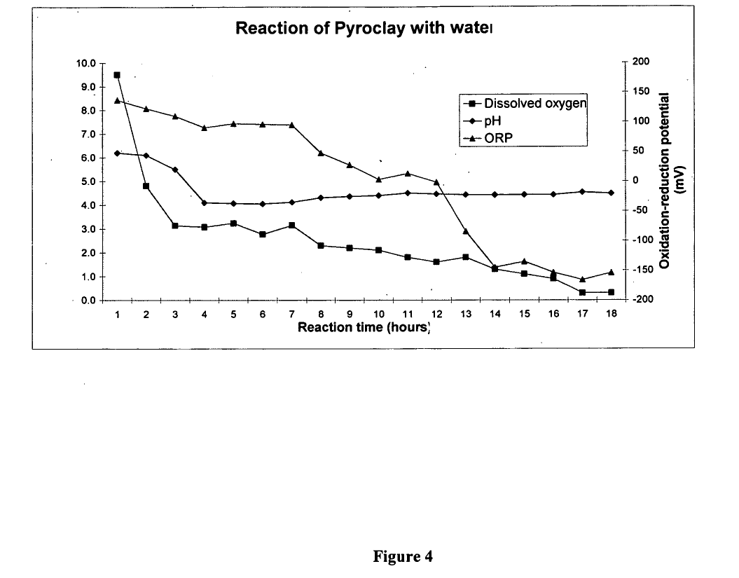

FIG. 4 shows the reaction of Pyroclay with water;

FIG. 5(a) shows the effect of adding pyrite to Wyoming bentonite on Staphlococcus epidermis;

FIG. 5(b) shows the effect of adding pyrite to Wyoming bentonite on Escherichia coli;

FIG. 5(c) shows the effect of adding pyrite and calcite to Kinney clay on Escherichia coli; and

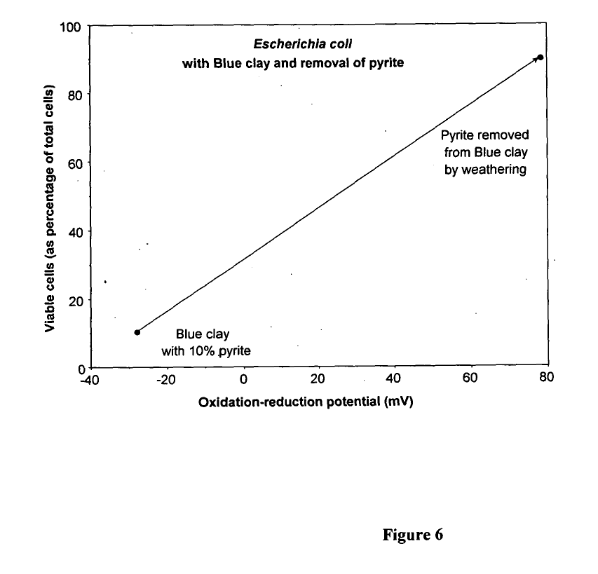

FIG. 6 shows the effect on Escherichia coli when pyrite is remove from Blue clay.

### DETAILED DESCRIPTION OF INVENTION

Various terms are used throughout the specification in describing the present invention. In order to provide a clearer and consistent understanding of the specification and claims, including the scope to be given such terms, the following definitions are provided.

Clay—“In general, the term clay implies a natural, earthy, fine-grained material which develops plasticity when mixed with a limited amount of water. By plasticity is meant the property of the moistened material to be deformed under the application of pressure, with the deformed shape being retained when the deforming pressure is removed. Chemical analyses of clays show them to be composed essentially of silica, alumina, and water, frequently with appreciable quantities of iron, alkalis, and alkaline earths.” (Grim, Clay Mineralogy, Second Edition, pp. 1-2 (1968)). The plasticity of clays generally results from a majority of their constituents having a very fine particle size (e.g., <2 micrometer equivalent spherical diameter). The plasticity property of clays is important in the present invention, because the compositions of the invention are hydrated so as to form a paste, and the hydrated composition then applied. Some natural clays are composed of a single clay mineral, whereas others may contain a mixture of clay minerals. In addition, clays may contain non-clay minerals, such as quartz, feldspar, calcite, pyrite, organic material and salts. Natural and synthetic clays may be employed within the scope of the invention. Reference herein to clays, unless specified differently, includes both natural and synthetic clays.

Clay minerals—Clay minerals are very fine-grain minerals (generally <2 micrometer equivalent spherical diameter) having the phyllosilicate structure. Because they are minerals, each type of clay mineral is defined by a certain crystal structure, which is, in the general case, the phyllosilicate-type structure, and each type of clay mineral has a limited range of chemical composition. A definition of the phyllosilicate structure (Bailey, 1980) states, “Clay minerals belong to the family of phyllosilicates and contain continuous two-dimensional tetrahedral sheets of composition T2O5 (T=Si, Al, Be, . . . ) with tetrahedra linked by sharing three corners of each, and with the fourth corner pointing in any direction. The tetrahedral sheets are linked in the unit structure to octahedral sheets, or to groups of coordinated cations, or individual cations.” In other words, “clay minerals” are a class of fine grain chemical compounds having the phyllosilicate structure, whereas “clays,” by contrast, are defined by their physical properties (e.g., plasticity), although they most often contain a substantial amount of clay minerals. Natural and synthetic clay minerals may be employed within the scope of the invention. Reference herein to clay minerals, unless specified differently, includes both natural and synthetic clay minerals.

Clay-like properties—Reference is made herein to materials having clay-like properties or the property of clays. For example, this phrase is used to describe the properties of the compositions within the scope of the invention. For the purposes of the present invention, a composition or material having clay-like properties is one that is fine-grained (having a maximum particle size of approximately 2 microns), and that develops plasticity when mixed with a limited amount of water or other suitable aqueous solution.

Paste—The term paste is used with reference to the form in which the composition within the scope of the present invention is used. The dry synthetic antibacterial clay composition within the scope of the present invention is hydrated to form a composition having the consistency of a paste. One skilled in the art would select a suitable amount of a pharmaceutically acceptable aqueous solution (i.e., water or saline) to add to the dry composition to provide a composition having the desired paste-like consistency. The specific consistency of the composition in the form of a paste is not critical so long as the hydrated composition, when in use, provides the desired properties described herein—i.e., acts as a barrier to atmospheric oxygen preventing it from entering the treated area; acts to effectively disperse the reducing agent therein; etc.

Processed—This term is used with reference to natural clays and compositions containing natural clay in particular, and refers to the processing of these to render them suitable for new and novel topical pharmaceutical use. For example, since natural clay samples are not pure clay minerals, processing may be necessary—i.e., clay-sized particles of <2.0 μm in diameter might be collected, organic material removed, the samples washed and sterilized using uV or heat to render any pathogenic virus, bacteria or protest noninfectious.

Suitable aqueous liquid—This phrase is used with reference to a liquid that may be used for hydrating the compositions within the scope of the present invention to create the new and novel pastes thereof. It is intended that these liquids be pharmaceutically acceptable/suitable for the use intended herein. Use of water is preferred.

Applicants' extensive research and study as to the antibacterial properties of many different natural clays resulted in their identifying a property which renders certain natural clays to have natural bactericidal properties. In particular, applicants identified that it is the presence of a bactericidal amount of particular reducing agents (i.e., pyrite, marcasite, Fe²⁺) in these natural clays that renders them bactericidal. The effectiveness of the reducing agent is related to its solubility and reactivity, which, in turn, is influenced by its particle size. They have found what they consider to be a mechanism behind its action. Applicants are now able to artificially produce a bactericidal composition having clay-like properties (referred to herein as a synthetic bactericidal (or antibacterial) composition), wherein said composition can be customized so as to illicit the properties desired. These compositions may have some advantages over simply using a natural antibacterial clay. For example, the artificially produced clay composition can be made purer (i.e., use of synthetic pyrite in place of natural pyrite, because naturally occurring pyrite may contain arsenic, selenium, cadmium or other toxic impurities, and use of a synthetic smectite as opposed to a natural smectite, as it could be rendered free of toxic metals) than naturally occurring clays, and its properties (i.e., reducing agent dissolution rate) can be optimized for the type of infection to be treated (i.e., the type of bacteria to be killed). Applicants' findings provide guidance useful in making synthetic bactericidal compositions having clay-like properties.

The present invention discloses synthetic bactericidal compositions having the properties of clay (clay-like properties), wherein these compositions comprise an bactericidal effective amount of a reducing agent and a clay, clay mineral or material having clay-like properties, wherein the reducing agent present in the composition is responsible for rendering the composition bactericidal.

The invention further discloses a method of topically treating bacterially-caused skin infections/diseases using these compositions. In use, water, or other suitable pharmaceutically acceptable aqueous liquids, such as sterile saline or phosphate buffered saline, is added to these compositions so as to create a paste (composition having a paste-like consistency). It is a composition in the form of a paste that is applied to the affected area for treatment.

Applicants have identified reducing agents that are responsible for rendering compositions within the scope of the present invention bactericidal. Suitable reducing agents that may be employed herein are the polymorphs of FeS₂, which include pyrite and marcasite, pyrrhotite, manganese oxides, FeS, FeSO₄, and other minerals or compounds that contain soluble reducing transition metals with like properties. They are used to remove oxygen from the treated site and produce chemical radicals (i.e., hydroxyl, nitrogen, or oxygen). We speculate that peroxide may be produced at, for example, the pyrite surface, and that this peroxide participates in a Fenton reaction with the ferrous iron to produce hydroxyl radicals that then attack bacterial cell walls, thereby killing the bacteria. [References: Cohn et al., “Role of pyrite in formation of hydroxyl radicals in coal: possible implications for human health,” Particle and Fibre Toxicology, 3: 16 (2006); Cohn et al., “Pyrite-induced hydroxyl radical formation and its effect on nucleic acids,” Geochemical Transactions, 7:3 (2006); Cohn et al., “RNA decomposition by pyrite-induced radicals and possible role of lipids during the emergence of life,” Earth and Planetary Science Letters, 225, 271-278 (2004)].

Applicants contend that the identification of the particular mineralogic make-up of the particular non-antibacterial or antibacterial clays used in the figures described below, while within the scope of this invention, is not the focus of the showing herein. The focus is to illustrate that the addition of pyrite to non-antibacterial clays renders them antibacterial, and that the removal of pyrite from antibacterial clays renders them non-antibacterial.

As shown in FIGS. 1(a) and 1(b) viable and dead or injured bacteria were distinguished microscopically. The top photomicrograph is of stained E. coli that were subjected to leaching in dialysis tube experiments with a non-antibacterial clay (Kinney montmorillonite), whereas the bottom photomicrograph shows the results of the same type experiment, but carried out with the Kinney montmorillonite containing 10% added pyrite. Clearly, pyrite makes the system antibacterial.

FIGS. 2(a), 2(b) and 2(c) provide graphical representations of the percentage of viable bacteria plotted as a function of the oxidation reduction potential (ORP) of the clay suspensions. These data were collected from 24-hour-experiments that were conducted in sealed enclosures that contained bacterial cultures in dialysis tubes surrounded by clay suspension (approximately 40 mg clay/mL water). The dialysis tubes had pores of a size through which solution could pass, but not bacteria or clay. Note that different bacterial species exhibited considerably different degrees of viability depending upon the clay type used. The clay types used are listed in Table 4, which also summarizes the experimental data used to draw FIGS. 2(a) through 2(c). Note also that antibacterial clays kill approximately 90% of the bacteria. In FIGS. 2(a), 2(b) and 2(c), R̂2 signifies the coefficient of determination, R².

FIG. 3 indicates the effect of Pyroclay concentration on E. coli viability and on oxidation-reduction potential for experiments lasting 24 hours. The most concentrated clay suspension, which contained 40 mg clay/mL water, was most effective for killing bacteria. The exponential nature of the regression curve in the figure suggests that increasing the clay concentration beyond 40 mg/mL would have little additional affect on killing power in these experiments.

FIG. 4 shows the results of an experiment in which a suspension of Pyroclay (40 mg clay/mL water) was monitored for a 24 h period by taking measurements of pH, dissolved oxygen (probe and colorimetric), and oxygen-reduction potential (ORP). Significant to this experiment was the rapid drop in DO and pH, accompanied by a more gradual development of reducing conditions signified by negative ORP values. The killing power of antibacterial clays is related to the decreasing value of these variables.

FIGS. 5(a), 5(b) and 5(c) provide graphical representations of the effect of pyrite addition and pyrite grind time upon bacterial (Staphylococcus epidermidis and E. coli) viability during dialysis tube experiments with two smectites (Wyoming and Kinney; see Table 3). These smectites, in their pure form, are not particularly effective antibacterial agents (Table 4). But they become antibacterial when a modest weight (1% or 10%) of pyrite is added. Antibacterial activity, determined by cell viability, increased a minimum of 2 to a maximum of 28 times that found for the pure clay minerals, the amount of increase depending on the clay mineral type, the bacteria type, the amount of pyrite added, and the grinding time for the pyrite. Applicants concluded that it is the addition of pyrite to non-antibacterial clays that produced levels of antibacterial activity similar to those observed for antibacterial clays (see Table 4). The effectiveness of the bactericidal agent, in this case pyrite, as measured by percent cell viability, is related to its reactivity and solubility, which, in turn, is influenced by its particle size, and therefore by grinding time. The addition of 10% calcite, added as a pH buffer, to the Kinney montmorillonite+10% pyrite system led to a decrease in antibacterial activity compared with that found for Kinney montmorillonite+pyrite systems.

These reducing agents are employed in the present invention in fine particulate form. Applicants have found that the finer the particle size, the more effective the reducing agents are, and the quicker the bactericidal activity occurs. For example, applicants ground pyrite for various periods of time, and found that, in general, the longer the grind, the more effective the antibacterial activity. See, for example, FIG. 5(b). This longer grinding time overall produced smaller pyrite particle size. Suitable particle sizes for the reducing agents herein may range from the nanometer to approximately 1 micron size range—and preferably less than approximately 1 micron in size. In a preferred embodiment, it is best for the reducing agent to be well mixed/dispersed in the compositions herein.

The reducing agents are present in the compositions herein in a bactericidal effective amount. These amounts may range from approximately 0.5% wt. to 10% wt., and preferably about 3% wt. of said composition. Applicants have determined that the presence of trace amounts, even less than 0.1% wt., of pyrite, for example, may render the compositions herein bactericidal. Trace amounts of pyrite were detected in the original sample of Argicur clay used to successfully treat Buruli ulcer in the Ivory Coast, West Africa. Of note is that for a given particle size, the more pyrite, for example, present, the stronger the bactericidal effect (see FIG. 5c); and for a given amount of pyrite generally the smaller the particle size, the stronger the bactericidal effect (see FIG. 5(b)).

FIG. 6 provides a graphical representation of the effect on bacterial viability of pyrite removal from an antibacterial clay. The antibacterial Blue clay naturally contains about 10% by weight pyrite (Table 1). But weathered Blue clay, which was collected from an oxidized zone above the Blue clay deposit, and which presumably was formed by the weathering of Blue clay, contains no pyrite and has little to no antibacterial properties (Table 4). We conclude that it is the pyrite that gives the Blue clay its antibacterial properties.

Because reactions involving, for example, pyrite oxidation may generate sulphuric acid, there is an optimal amount of pyrite, depending on its particle size, that may be present in a composition herein. With too much pyrite, the acid generated may mildly burn the skin tissue. For example, natural antibacterial clay from Oregon (Blue clay in Table 1) was found to contain pyrite that is nano-size (by transmission electron microscopy, TEM), and contains approximately 10% by weight pyrite in bulk. This clay was found to be bactericidal and to produce fairly acidic leachates (H₂SO₄, pH 2-2.5). Application of this clay in an unbuffered solution might create a pH condition damaging to human or mammalian skin tissues. Therefore, although the addition of large quantities of a reducing agent such as pyrite, for example, into a composition such as that described herein would still render the composition effective as a bactericidal composition, the presence of larger quantities of pyrite would not be desirable. We are currently experimenting with adding a pH buffer, such as calcite, to clay-pyrite compositions (see description as to mediation of pH and ORP by calcite addition set forth under Brief Summary of the Invention, herein, and diagramed in FIG. 5(c)). Thus it is within the scope of the present invention to balance the addition of such a buffer that will remove acidity but not significantly influence the invention's bactericidal activity. In addition to the undesirable pH, the addition of large quantities of pyrite, for example, would also render the composition unpleasant to apply to the skin. The addition of pyrite could be avoided altogether if bactericidal effective amounts of reducing ions, such as Fe²⁺, for example, were incorporated directly into the clay mineral structure, or were present as exchange ions on the clay.

It is also within the scope of the present invention to incorporate one or more reducing agents in the compositions herein. Should more than one reducing agent be employed, one skilled in the art will recognize that the combined amount of these would be such as to have a combined bactericidal effect—and, hence, the combined amount would translate as being a bactericidal effective amount present in the composition.

In a preferred embodiment, the fine particulate size, as well as the amounts of the reducing agent present in the composition of the invention, is such as to not materially affect the clay-like properties of the composition.

The preferred reducing agent is pyrite. Course grained, naturally occurring, pyrite may be ground and employed herein. As an alternative, pyrite can also be synthesized to be very fine. See Ohfuji et al., “Experimental syntheses of framboids—a review,” Earth Science Reviews, 71, pp. 147-170 (2005) and Shi et al., “Synthesis, characterization, and manipulation of dendrimer-stabilized iron sulfide nanoparticles,” Nanotechnology, 17, pp. 4554-4560 (2006). The teachings of these references are incorporated herein by reference.

Clays serve in the compositions herein as pharmaceutically acceptable carriers for the reducing agent. In addition to serving as a suitable carrier for the reducing agent, wherein the reducing agent may be thoroughly dispersed therein, the clays herein also (a) serve as an effective low permeability barrier to keep atmospheric oxygen from the skin or other surface to be treated, (b) serve to render compositions within the scope of the invention to have clay-like properties, (c) serve to keep the site/system to be treated moist, while absorbing excess liquid, and (d) aid in the dissolution of the reducing agent through the process of ion exchange, an effect that is described for the dissolution of sparingly soluble compounds by montmorillonite in Eberl and Landa, “Dissolution of alkaline earth sulfates in the presence of montmorillonite,” Water, Air, and Soil Pollution, 25: 207 (1985). The teachings of this reference are incorporated herein by reference.

It is also within the scope of the present invention that the final synthetic bactericidal composition have the properties of a clay. Suitable clays that may be employed herein include smectite-clays, illite-clays, rectorite-clays, other clays having like properties, or mixtures thereof. These clays may be natural or synthetic. In addition to clays, applicants contend that polymers or other materials having clay-like properties (fine particle size, and plasticity) may be suitably employed for the clays herein. One having ordinary skill in the art will recognize the purpose for which the clays are employed; and, accordingly, will recognize other suitable materials that may be used in its place. Applicants contend that these materials are included within the compositions claimed herein.

Should a composition within the scope of the present invention comprise a natural clay, one having ordinary skill in the art will recognize that there may be a need to process the natural clay so as to render it suitable for the purpose described herein—topical pharmaceutical use. How to process natural clays so as to render them suitable for pharmaceutical use is well within the skill of the art.

We speculate that certain clay minerals which contain ferrous iron (Fe⁺²) in their structure may not need to have a reducing agent added to demonstrate bactericidal properties (e.g., some smectite clay minerals have elevated Fe⁺² content, but no pyrite). The existence of such iron (or other reduced transition metal) in the clay itself may fulfill the role, for example, of the pyrite. Ferrous iron, for example, could be present in the octahedral sheet of the clay mineral, or as an exchange ion in its interlayer, and act as an effective reducing agent. Octahedral ferrous iron present in smectite would be close to the solution, because the smectite particles are so thin (approximately 1 nm), and therefore could participate in oxidation-reduction reactions.

### Clays and Materials Having Clay-Like Properties

### Smectite Clay

Smectite is a general name used for swelling clay that has approximately 1-nm thick 2:1 layers (c-direction of unit cell) separated by hydrated interlayer cations which give rise to the clay's swelling. The “a” and “b” dimensions of the mineral are on the order of several microns. The layers themselves are composed of two opposing silicate sheets, which contain Si and Al in tetrahedral coordination with oxygen, separated by an octahedral sheet that contains Al, Fe and Mg in octahedral coordination with hydroxyls. These 2:1 layers (two tetrahedral sheets with an octahedral sheet in between) carry a net negative charge that is balanced by interlayer cations. Because the surfaces of the 2:1 layers are charged, they attract cations and water, which leads to swelling. The operational definition is that smectite swells to give a 17 Angstrom-unit x-ray diffraction peak when treated with ethylene glycol. Because the 2:1 layers are less than 1 nm thick, and because the interlayer is open to solution, smectite has very special properties. For example, it has an enormous surface area of approximately 750 m² per gram, can absorb polar organic and other molecules in the interlayers, and can exchange interlayer cations with the solution. It can further protect interlayer organics from bacteria and oxygen, and can aid in the dissolution of insoluble substances through its exchange properties. Due to its very fine particle size, it can be impermeable to gases. In addition, it has catalytic properties important for many organic reactions as well.

There are a number of different types of smectite, which are classified with respect to the location of the negative charge on the 2:1 layers, and based on the composition of the octahedral sheet (either dioctahedral or trioctahedral). Dioctahedral smectites include beidellite (majority of charge located in tetrahedral sheet) and montmorillonite (majority of charge in octahedral sheet). Similar trioctahedral smectites are saponite and hectorite. Swelling and other properties of smectite can be altered by exchanging the dominant interlayer cation. For example, swelling can be limited to 2 water layers by exchanging Na for Ca.

Smectites are well known in the art and are available commercially from a variety of sources—for example, from WyoBen Co. (Greybull, Wyoming) and American Colloid (now called AmCol, Arlington Heights, Ill.). In addition, they can be synthesized, and can be found in large deposits, particularly in the American western deserts—i.e., the Cheto clay in Arizona. Smectites are frequently found in nature mixed with impurities (i.e., calcite and quartz), but, due to the very fine particle size, can be purified by size fractionation in water. During this process, the coarser grained minerals (i.e., quartz and calcite) settle out, and the smectite is poured off with the aqueous suspension and dried.

Numerous methods exist for synthesizing smectite. For example, U.S. Pat. No. 4,861,584 (Powell, et al.) sets forth a description of smectite-type clays (see col. 4, lines 37 through col. 5, line 55) and identifies that smectite clays can be prepared synthetically by either a pneumatolytic or a hydrothermal synthesis process. Powell et al. provides a list of USPs as describing representative hydrothermal processes for preparing synthetic smectites. USP '584 further describes at col. 5, lines 40+ a hydrothermal process for synthesizing smectite clays. The teachings set forth in Powell, et al., as well as the teachings set forth in the list of USPs identified in Powell et al., are incorporated by reference herein in their entirety.

It is within the scope of the present invention to utilize the various types of smectite in the compositions of the present invention.

### Illite Clay

It is also within the scope of the present invention to utilize illite-clay in the compositions of the present invention. Illite, though similar to smectite, is a non-swelling clay. It has its 2:1 layers bound together by K ions so that it does not swell.

### Mixed-Layer Clay

Oregon Blue clay, for example, is composed of an ordered mixed-layer illite/smectite (referred to as K-rectorite). It has regular alternation of illite and smectite layers parallel to the “c” axis. It combines the swelling properties of smectite with non-swelling illite.

Suitable clays, for example, such as smectite, illite, mixed layer clays such as rectorite, and other suitable clays may be synthesized using conventional methods well known in the art. In general, one skilled in the art would simply start with a gel or glass having the chemical composition of the clay to be synthesized, and heat it in water using a pressure vessel. (For example, experimental variables, such as temperature, time and pressure required to synthesize smectite and various forms of rectorite are described in Eberl, “Reaction series for dioctahedral smectites,” Clays and Clay Minerals, Vol. 26, 327-340 (1978)). Suitable clays should be very fine grained, hold water well, and be absorptive.

Examples of synthetic clays and clay minerals that may be suitable for use herein include, but are not limited to, synthetic hectorite, which is a layered hydrous magnesium silicate known as Laponite^{R} (Southern Clay Products, Gonzales, Tex.), a synthetic mica-montmorillonite, such as Barasym^{R} (Baroid Division, NL Industries, Houston, Tex.) and mixtures thereof. Useful natural types of clays include swelling clays such as aliettite, beidellite, nontronite, saponite, sauconite, stevensite, swinefordite, volkonskoite, yakhontovite, hectorite, montmorillonite, bentonite and mixtures thereof. [Note U.S. Pat. No. 6,015,816 (i.e., col. 4, lines 10+)—the teachings of USP '816 are incorporated by reference. This U.S. Pat. No. 6,015,816 also references examples of specific types of clays from the smectite mineral group as including: hectorite (“SHCa-1”—Source Clay Minerals Repository, University of Missouri, Columbia, Mo.), Cheto montmorillonite (“SAz-1”), etc.]. In addition, 1:1 clay minerals such as kaolinite and serpentine, as well as 2:1:1 clay minerals such as chlorite may also prove to be useful, as may chain-type clays such as palygorskite and sepiolite.

Selection of clays and/or materials having clay-like properties that would be suitable for use in the present invention is well within the skill of the art in light of the description herein.

Compositions within the scope of the present invention may be made by mixing one or more selected reducing agents with a selected clay, clay mineral or material having clay-like properties so as to disperse the reducing agent(s) therein. Ideally, the reducing agent(s) is uniformly dispersed throughout the synthetic composition.

The compositions herein are believed useful to topically treat infections and skin diseases caused by various types of bacteria such as, for example, Staphylococcus aureus (MRSA and non-MRSA), Pseudomonas aeruginosa, Streptococcus sp., Mycobacterium ulcerans, E. coli, ESBL E-coli, Salmonella typhimurium, Staphylococcus epidermidis, M. smegmatis, and M. marinum. Treatment of these infections is accomplished by hydrating the composition herein so as to form a paste, and then topically applying the paste to the affected area as well as to portions of the surrounding area. More than one, and perhaps a series of applications may be needed to treat the affected area. A clay compress, poultice, or paste should be washed off and/or changed daily. In addition, applicants believe that the compositions herein may also possibly be useful to treat protest skin infections such as Leishmania, and possibly viral skin infections as well.

The presence of reducing agents, such as pyrite, in the synthetic bactericidal compositions of the invention is essential for rendering said composition bactericidal. At this point, applicants can only speculate as to the role played by the clays described herein. Applicants postulate that these types of clays can (1) absorb toxins, (2) absorb Fe⁺² dissolved from the reducing agent (i.e., pyrite), (3) perhaps absorb and preserve H₂O₂ released by the Fenton reaction, and (4) keep oxygen away from the reaction so that Fe⁺², for example, from the reducing agent, is not immediately oxidized. (See “Proposed Theory of Activity,” outlined below.) In addition, the clays or materials having clay-like properties of the type described herein are excellent carriers for the reducing agents, are normally very soothing to the skin, and able to keep the system moist because they absorb water.

### Proposed Theory of Activity

The reducing agent is essential to the composition of the present invention. Without wishing to be bound by theory, it is applicants' belief that the compositions within the scope of the present invention are antibacterial due to perhaps one, or more of the following activities:
(a) the removal of oxygen from the site of infection—depriving the bacteria of oxygen;
(b) the production of peroxide at the surface of the reducing agent, i.e., pyrite; and/or
(c) the production of hydroxyl radicals (.OH) by the Fenton reaction.
   Applicants believe that these activities are all related to the presence of Fe⁺², for example, in the reducing agent (i.e., pyrite) and in some cases other reduced forms of transition metals (e.g., Mn, Cr, Zn, Cu) may substitute and produce a similar antibacterial reaction. It is believed that the invention deprives the bacteria of oxygen and may produce hydroxyl radicals, thereby causing disruption of bacterial cell membrane proteins and lipopolysaccharides. Applicants have learned that the finer the particle size of pyrite, for example, the faster the reaction—oxygen is removed faster and more completely.

A possible mechanism of how it is believed this theory might operate can be described as follows in relation to the use of pyrite as the reducing agent:

Some of the pyrite present in the composition oxidizes so as to remove oxygen from the system (from the infected wound). Dissolution of the pyrite buffers the oxygen at a low level. It is believed that the clays or materials having clay-like properties described herein (i.e., smectite clay), and in which the pyrite is dispersed, prevent additional oxygen (atmospheric oxygen) from entering the system, and hence not allowing replacement of the oxygen that has been removed by the above reaction. By varying the thickness of the applied paste, clay composition, and degree of water-clay balance, for example, the entry of oxygen into the system can be modified or controlled and the proposed reactions (see below) maintained. In addition, the clay itself may further sorb toxins released by the bacteria. With essentially no oxygen in the system, water present in the clay reacts at the pyrite surface to form peroxide (H₂O₂). It is believed that the reaction of water at the pyrite surface keeps H₂O₂ at a steady concentration. Fe⁺² from the pyrite then reacts with the peroxide to form hydroxyl radicals (.OH) by the Fenton reaction (see Eq. 1 and Eq. 2):

Fe⁺²+H₂O₂→Fe⁺³+.OH+OH⁻ (Eq. 1)

Fe⁺³+H₂O₂→Fe⁺²+OOH.+H⁺ (Eq. 2)

In the net reaction, the presence of iron is truly catalytic and two molecules of hydrogen peroxide are converted into two hydroxyl radicals and water—description obtained from http://en.wikipedia.org/wiki/Fenton's reagent.

Applicants believe that Fe⁺² keeps up the steady production of hydroxyl radicals. It is further believed that these hydroxyl radicals then react with the bacterial cell wall, hence killing the bacteria. Within this theory, it is believed important that the peroxide (H₂O₂) be released slowly so as to not decompose into oxygen. The low oxygen level is buffered by the reducing agent; therefore, if additional oxygen enters the system, the reducing agent serves to remove it. Likewise, the peroxide may be buffered by reaction at the pyrite surface. Although the role played by the pH of the composition is not specifically known, it is believed that the pH should optimally range from approximately between 3 and 6. If the pH of the composition were to rise above this approximate range, it would likely be difficult to maintain iron in the ferrous (Fe⁺²) state, and peroxide may decompose to oxygen. Finally, a pH below this range may damage skin or other tissue(s).

In addition to clays such as smectite serving to prevent atmospheric oxygen from entering the system, the clay serves to keep the system moist. This is believed to be important because water is needed to produce the peroxide as set forth above. Applicants further believe that the clays or materials having clay-like properties may, perhaps, also absorb toxins and thus possibly reduce the virulence factor/effect of the infection. Clay minerals also may attract Fe²⁺ from the pyrite and hold it as an exchange ion, thereby rendering it more reactive than when it is held in the pyrite structure.

Described more generally, it is believed that the mechanism(s) of action appear to be bacterial death or viability loss caused by a lowered oxidation-reduction potential (reducing conditions), a several to many fold reduction in dissolved oxygen level, and/or a concomitant lowered pH. Applicants have found that bacteria viability was affected by (1) variation in pyrite content, (2) pyrite grain/particle size, and (3) degree of redox potential.

The reaction takes place in a matter of hours and reaches steady state within 24 hours. Time series data using leachates of the Oregon blue clay show killing occurs over the same time interval that it takes for the oxidation-reduction potential (ORP) to reach steady state. Perhaps it is the gradient, meaning the rapid ORP change over time, that the bacteria cannot accommodate.

### Experimental Procedures and Data

### In General:

Through carefully controlled experiments with different bacteria physically separated from clay containing suspensions (suspensions of an antibacterial clay composition within the scope of present invention) within dialysis tubing (see description set forth in Dialysis Tube Experimentation, below), a somewhat rapid abiotic/chemical reaction (within a few hours) takes place whereby pH and dissolved oxygen within the culture system are significantly reduced. In addition, experimental evidence illustrates that lowered pH, oxidation-reduction potential (ORP) and oxygen all accompany suspension of the bactericidal clay composition and water. This observation indicates that the reaction is spontaneous and occurs in the absence of bacteria. Further laboratory experimentation with dialysis tubes containing bacterial cultures with this suspension indicates that (1) bacteria are either killed or rendered nonviable (confirmed by both plate, and by microscopic evaluation with specific stains designed to measure cell membrane integrity), and (2) the addition of small amounts of pyrite to previously non-bactericidal clays produces a similar effect (lowered ORP and oxygen) and similar bactericidal capability. A description of these experiments is set forth herein, and results are shown in Table 4.

### Dialysis Tube Experimentation

In order to assess if there were compounds or chemicals in the leachate of a clay or clay containing composition, or if its bactericidal activity were maintained, a dialysis tube experimental series was designed to separate, and keep separate, a clay or clay containing composition being evaluated and a bacteria in liquid culture. For example, Applicants previously leached bactericidal clay compositions to see if toxic elements, such as As, Pb or Ag, were present, but nothing stood out. The methodology used is as follows:

Bacteria, grown to log phase to approximately 1×10⁹ cells/mL in liquid culture, were diluted to 1×10⁶/mL (initial bacterial concentration) in sterile media. Bacterial suspensions were filter-concentrated to 0.22 μm using black polycarbonate filters [GE Osmonics PCTE (polycarbonate track-etch), catalog K02BP02500], and enumerated by epifluorescence microscopy using DAPI (4′,6-diamidino-2-phenylindole, Sigma Aldrich, St. Louis, Mo.) direct counting procedure. General protocols were modified from Hobbie et al. [Hobbie et al., “Use of nuclepore filters for counting bacteria by fluorescence microscopy,” Applied and Environmental Microbiology, 33: 1225-1228 (1977)] as described in Harvey et al. [Harvey et al., “Effect of organic contamination upon microbial distributions and heterotrophic uptake in a Cape Cod, Mass. Aquifer,” Applied and Environmental Microbiology, 48: 1197-1202 (1984)], and Metge et al. [Metge et al., “Analysis of free-living microbial abundances and size distributions,” Report ID MA-0025, U.S. Geological Survey, WRD (1997)].

Dialysis tubes (Spectrum Laboratories, Inc., catalog 235057, Rancho Dominguez, CA) with a 25000 kilodalton (kDa) molecular weight cut-off (MWCO) were used for all analyses except when marcasite was present as a reducing agent in a bactericidal clay when 20000 MWCO (Spectrum Laboratories, Inc., catalog G235057) was used instead. Dialysis tubes, in all cases, were washed at least three times with filtered, autoclaved 1 mM NaCl solution, subjected for several hours to uV light to further sterilize them within a biosafety level II hood. Tubes were rewashed with sterile 1 mM NaCl before clay addition.

Freshly-prepared clay-containing suspensions (40 mg/mL final concentration—clay or clay containing composition within the scope of the present invention suspended in sterile distilled water or 1 mM NaCl, both at pH 5.6-6.0) were carefully loaded into the outer reservoir of the dialysis tube and left overnight to allow further development of stable redox (oxidation-reduction potential, ORP), oxygen, and pH levels. Bacteria cultures to be evaluated were then individually loaded into the interior sleeve, to replicate dialysis-tubing chambers, using sterile techniques, with the outer sleeves containing clay or mineral suspensions at approximately 40 mg/mL. The dialysis chambers containing separate bacteria and clay containing suspensions were sealed and placed on a rocker table with gentle agitation. This maintained a constant mixing of clay containing suspension in which bacteria in dialysis membranes were placed. After overnight exposure to clays or mineral suspensions, bacterial cultures within the dialysis chambers were removed, enumerated by either epifluorescence microscopy or by flow cytometry, and microbial viability assessed using applied staining techniques.

The viability testing employed a commercially available fluorescent staining technique. Viable and nonviable bacteria were determined by staining with Invitrogen (Carlsbad, Calif.) Live/Dead (V-7007 and V-7012) staining kits and suggested protocols. These stains allow differentiation between cells based upon membrane integrity and cell surface character through use of different applied epifluorescent stains. In live/dead assays that use an epifluorescence filter cube capturing FITC/TRITC and Texas Red Emission spectra, viable cells fluoresce green whereas dead or nonviable cells fluoresce red. Viable and nonviable cell levels were compared against standard curves generated for each bacterial isolate and the degree of bactericidal activity assessed. Additionally, pH and dissolved oxygen as well as ORP were measured at the beginning and conclusion of experiments to determine how or if basic chemical conditions changed.

### Experiments Conducted

### Example 1

Wyoming montmorillonite (SWy-1, a smectite clay which is sold as a standard by The Clay Minerals Society, Chantilly, Virginia), which is commonly used in industry as a drilling mud, was mixed with pyrite to prepare an artificial (synthetic), antibacterial composition having the property of a clay (clay-like property). The fraction of the clay used was the <1 micron size fraction, which was separated out by centrifugation. Chemical analysis via X-ray fluorescence (XRF) of the clay (Ca-saturated), prior to pyrite amendment, found the weight percent (fired basis) of major oxides therein, which are presented in Table 2.

The elements in Table 2 were cast into a structural formula (Table 3) by assuming an anion content of O₁₀(OH)₂, and by varying the ferrous/ferric ratio of the total iron until the formula balanced electrically. However, the exact clay used is not so important as long as it has the properties of fine grain size to keep out oxygen, and has the absorptive properties common to smectite. The important point is that the clay did not effectively kill bacteria (very effective antibacterial clay kills at least 90% of the bacteria) until the pyrite was added. To this clay, 10% wt. pyrite was added, and the resulting compositions tested.

The pure Wyoming montmorillonite, using the dialysis techniques, did demonstrate a slightly reducing environment and was somewhat bactericidal (approximately 19% viability as to E. coli). This activity is attributed to octahedral Fe²⁺ in the clay's structure (Table 2). However, bacterial viability was reduced by 2 fold when 10% weight of pyrite (ground 0.5 h) was added. When finer grained pyrite at 10% by weight was added, bacterial viability was reduced by 7-fold (see FIG. 5A, 5B, and Table 3).

### Non-Antibacterial Clay Trial

A smectite clay (Kinney montmorillonite; concentration 40 mg L⁻¹; chemical analysis in Table 2, and structural formula in Table 3) was tested upon log phase E. coli bacteria within dialyses tubes and using setup and methods as described previously. The treatments tested were Kinney alone, Kinney with 10% and 1% pyrite amendment, and Kinney with 10% pyrite and 10% calcite amendments. Bacteria were evaluated at ˜24 h using viability stains as described previously. Results are shown in Table 4 (below) and also in FIG. 5C (below). The solution pH and oxidation reduction potentials were measured at ˜24 hr as well. FIG. 1 is a photomicrograph taken with epifluorecent microscopy using illumination techniques that illustrates the viable and nonviable bacterial cells from the dialyses tubing after 24 h.

Similar experiments were conducted using Ormalite with E. coli—see Table 4. Ormalite is a clay found proximally to but not within the Blue clay formation. This clay was not antibacterial.

### CONCLUSION

It was apparent, from dialysis experiments with varied bacterial types, that the clay containing compositions are responsible for the bactericidal properties of the clays evaluated. There was a clear difference between bactericidal clays and non-bactericidal clays which could be monitored by redox potential measurement. Bactericidal clays were characterized by becoming reducing over 24 hours; likewise, there were differences in the degree of bacterial viability with bacterial cultures tested. This observation indicated that certain bacterial species were more susceptible than others to bactericidal clays.

Non-antibacterial clays or clay minerals can be rendered antibacterial in three ways: (1) by mixing a clay or a clay mineral with an antibacterial effective amount of a substance that contains a reducing agent, for'example, by mixing illite with pyrite; (2) by exchanging interlayer cations, or cations associated with edge hydroxyls, in clay minerals with an antibacterial effective amount of reducing ions, for example, by exchanging interlayer Na⁺ ions for Fe²⁺ ions in montmorillonite (methods for accomplishing such exchanges have been described, for example, by Hofstetter et al., 2003); and (3) by reducing cations already present in a clay mineral structure to produce an antibacterial effective amount of a reducing agent, for example, by reducing Fe³⁺ to Fe²⁺ in the octahedral sheet of nontronite using methods that have been described by Stucki et al. (1984).

Applicants believe that the synthetic bactericidal compositions within the scope of the present invention are effective in topically treating infections and/or skin diseases caused by numerous types of bacteria, including antibiotic-resistant bacteria. Bactericidal compositions within the scope of the present invention were found to have various degrees of bactericidal effectiveness, killing in 24 hours up to 99% of colonies of Staphylococci [both Staphylococci aureus (MRSA) and Staphylococci epidermidis], Escherichia coli, Salmonella typhimurium, and Pseudomonas aeruginosa.

As previously stated, for optimal effectiveness in accordance with the present invention, the reducing agents must be well mixed and evenly dispersed throughout the clay, clay mineral, or material having clay-like property employed.

One having ordinary skill in the art will recognize the potential advantages of synthesizing and using a composition within the scope of the present invention over the use of natural antibacterial clays. Some advantages may include greater purity and optimization of properties for a targeted use—i.e., optimization of pyrite dissolution rate so as to kill a specific type of bacteria.

As previously disclosed, one skilled in the art will recognize that a bactericidal effective amount of a reducing agent may be added to any natural clay, regardless as to whether or not that natural clay has natural bactericidal properties, so as to be certain that a composition containing that natural clay is suitable for the purpose intended herein. Prior to the present invention, applicants contend that there was no motivation to add a reducing agent to a clay for the use described herein.

In addition, the results of applicants' research provides guidance, not previously available, but very long sought, regarding how to identify a natural clay that would be suitable for topically treating bacterial infections. That guidance being that natural clays containing therein a bactericidal amount of a reducing agent, such as pyrite or marcasite, may be suitable for this purpose.

Although specific reference to pyrite is often used as the reducing agent in describing the invention herein, one having ordinary skill in the art will recognize that it is applicants' intent that the description herein not be so limited. Specific reference in the description to pyrite is made for exemplary purposes only. The description applies to use of the other identified reducing agents as well.

While particular embodiments of the present invention have been shown and described, it will be obvious to those skilled in the art that changes and modifications may be made without departing from this invention. Therefore, it is intended that the claims herein are to include all such obvious changes and modifications as fall within the true spirit and scope of this invention.

### REFERENCES

Bailey, “Summary of recommendations of AIPEA nomenclature committee on clay minerals,” American Mineralogist, 65: 1-7 (1980)
Brunet de Courssou, “Preliminary Studies of Debridement using Clay Minerals on Mycobacterium Ulcerans Infections and their Ramifications,” (Feb. 4, 2002)—printed on Mar. 26, 2009 from http://fifthkingdom.net/burulibusters/default.htm
Cohn et al., “RNA decomposition by pyrite-induced radicals and possible role of lipids during the emergence of life, Earth and Planetary Science Letters, 225, 271-278 (2004)
Cohn et al., “Role of pyrite in formation of hydroxyl radicals in coal: possible implications for human health,” Particle and Fibre Toxicology, 3: 16 (2006)
Cohn et al., “Pyrite-induced hydroxyl radical formation and its effect on nucleic acids,” Geochemical Transactions, 7:3 (2006)
Eberl and Landa, “Dissolution of alkaline earth sulfates in the presence of montmorillonite,” Water, Air, and Soil Pollution, 25: 207 (1985).
Eberl, “User guide to RockJock—A program for determining quantitative mineralogy from X-ray diffraction data,” USGS Open File Report OF 03-78, 40p (2003)
Harvey et al., “Effect of organic contamination upon microbial distributions and heterotrophic uptake in a Cape Cod, Mass. aquifer,” Applied and Environmental Microbiology, 48:1197-1202 (1984)
Haydel et al., “Broad-spectrum in vitro antibacterial activities of clay minerals against antibiotic-susceptible and antibiotic-resistant bacterial pathogens,” Journal of Antimicrobial Chemotherapy, 61, pp. 353-361 (2008)—(advance access publication 10 Dec. 2007).
Hobbie et al. 1977, “Use of nuclepore filters for counting bacteria by fluorescence microscopy,” Applied and Environmental Microbiology, 33:1225-1228 (1977)
Hofstetter et al., “Reactivity of Fe(II) species associated with clay minerals,” Environ. Sci. Technol, 37, 519-528 (2003)
Kostyniak et al., U.S. Pat. No. 6,015,816, “Antimicrobial Compositions,” (Jan. 18, 2000)
Metge et al. “Analysis of free-living microbial abundances and size distributions,” Report ID MA-0025, U.S. Geological Survey, WRD (1997)
Metge et al., “Development and implementation of bacterial isolates expressing blue and green fluorescing proteins (BFP and GFP) as alternatives to conventional DNA- and protein-specific fluorescence staining for studying bacterial transport behavior in granular media,” Geological Society of America Abstracts with Programs, 38:28 (2006)
Metge et al., “Bactericidal properties of clays used for treatment of Buruli ulcer—an emerging public health threat,” Geological Society of America Abstracts with Programs, 39:60 (2007)
O'Hanlon, “Medicinal clays may heal ulcers,” News in Science, (Oct. 26, 2007)—printed on Jan. 22, 2009 from http://www.abc.net.au/science/news/stories/2007/2071334.htm
Ohfuji et al., “Experimental syntheses of framboids—a review,” Earth Science Reviews, 71:147-170 (2005)
Shi et al., “Synthesis, characterization, and manipulation of dendrimer-stabilized iron sulfide nanoparticles,” Nanotechnology, 17:4554-4560 (2006)
Stucki et al., “Preparation and handling of dithionite-reduced smectite suspensions.” Clays and Clay Minerals, 32, 191-197 (1984)
Williams et al., “Chemical and Mineralogical Characteristics of French Green Clays Used for Healing,” Clays and Clay Minerals, 56:437-452 (2008)
Williams et al., “Killer Clays! Natural antibacterial clay minerals,” Mineralogical Society Bulletin, pages 3-8 (April 2004).
Williams et al., “Scientific validation of antibacterial minerals needed for public policy support,” Geological Society of America Abstracts with Programs, 38:367 (2006)
Williams et al., “Bentonite, Bandaids and Borborygmi, Elements, in press (2009)
Williams et al., “Evaluation of the medicinal use of clay minerals as antibacterial agents,” International Geology Review, in press (2009)
“Scientists Discover Clays to Fight Deadly Bacteria,” Infoniac (Oct. 29, 2007)—printed on Jan. 22, 2009 from http://www.infoniac.comiscience/scientists-discover-clays-fight-deadly-bacateria.html

TABLE 1
Unpublished mineralogical composition of clays (in weight %) used in antibacterial experiments.

| Sample name | | | | | | |
|---|---|---|---|---|---|---|
| Argicur | Pyroclay | Blue clay | Weathered Blue | Argiletz | Miraculite | Ormalite |
| (ABC) | (ABC) | (ABC) | (NABC) | (NABC) | (NABC) | (NABC) |

NON-CLAYS

| | | | | | | | |
|---|---|---|---|---|---|---|---|
| Quartz | 1.2 | 43.2 | 44.6 | 46.0 | 18.2 | 11.1 | 13.8 |
| Feldspar | 6.8 | 1.9 | 0.9 | 2.9 | 5.6 | 12.0 | 0.0 |
| Amphibole | 0.0 | 0.0 | 0.5 | 0.0 | 0.0 | 0.0 | 0.0 |
| Pyrite | trace | 3.4 | 9.6 | 0.0 | 0.0 | 0.0 | 0.0 |
| Barite | 0.0 | 1.0 | 0.0 | 0.0 | 0.0 | 0.0 | 0.0 |
| Gypsum | 0.0 | 1.2 | 0.0 | 0.9 | 0.0 | 1.9 | 0.0 |
| Anatase | 0.0 | 0.0 | 0.0 | 0.2 | 0.0 | 0.0 | 0.7 |
| Jarosite | 0.0 | 0.8 | 0.2 | 0.0 | 0.0 | 8.7 | 0.0 |
| Magnetite/maghemite/ | 0.0 | 1.0 | 0.0 | 0.0 | 0.0 | 5.0 | 0.0 |
| goethite | | | | | | | |
| Calcite | 5.7 | 0.0 | 0.0 | 0.0 | 17.8 | 0.0 | 0.0 |
| Total non-clays | 13.7 | 52.5 | 55.8 | 50 | 41.6 | 38.7 | 14.5 |

CLAYS

| | | | | | | | |
|---|---|---|---|---|---|---|---|
| Kaolinites | 0.0 | 0.4 | 0.7 | 2.9 | 0.9 | 0.0 | 5.5 |
| Illite + muscovite | 49.2 | 9.7 | 7.6 | 30.9 | 43.3 | 17.2 | 78.7 |
| Chlorite | 0.0 | 3.2 | 4.4 | 0.8 | 4.5 | 0.0 | 0.0 |
| Rectorite | 0.0 | 26.2 | 48.4 | 24.9 | 0.0 | 0.0 | 1.7 |
| Smectite | 33.9 | 0.0 | 0.0 | 0.0 | 18.5 | 46.7 | 0.0 |
| Pyrophyllite | 0.0 | 2.9 | 0.0 | 0.0 | 0.0 | 0.0 | 0.0 |
| Gibbsite | 0.0 | 0.0 | 0.0 | 0.0 | 0.0 | 1.2 | 0.0 |
| Total clays | 83.1 | 42.4 | 61.1 | 59.5 | 67.2 | 65.1 | 85.9 |
| Total | 96.8 | 94.9 | 116.9 | 109.5 | 108.8 | 103.8 | 100.4 |

ABC = antibacterial clay; NABC = non-antibacterial clay. Clays were analyzed by quantitative X-ray diffraction using the RockJock computer program (Eberl, 2003).

TABLE 2
Chemical analyses, byX-ray fluorescence (XRF) spectrometry,
and based on weight when fired to 925 C, of the <1 micron
size fraction of pure smectites used in experiments.

| | Wyoming montmorillonite | Kinney montmorillonite |
|---|---|---|
| Oxide | Weight % oxide | Weight % oxide |
| SiO2 | 66.44 | 66.87 |
| Al2O3 | 22.93 | 22.40 |
| Fe2O3 | 4.48 | 1.42 |
| MgO | 2.91 | 5.25 |
| CaO | 3.27 | 0.06 |
| K2O | 0.07 | 0.03 |
| Na2O | 0.45 | 4.38 |
| TiO2 | 0.11 | 0.13 |
| Total | 100.66 | 100.54 |

TABLE 3
Structural formulae for pure smectites (<1 micron size fractions) used in the
experiments, calculated from the data in Table 2. The first parentheses in the formulae below
contain the octahedral cations, the second the tetrahedral cations. The first brackets contain
the 2:1 layer atoms, with the net negative charge as the superscript. The second brackets
contain the interlayer cations, with their balancing positive charge as the superscript. These
clay minerals also contain loosely bound water molecules (nH₂O), which are not included in
the formulae.

| Clay mineral | Structural formula |
|---|---|
| Wyoming | [(Al_{1.52}Fe_{0.07}³⁺Fe_{0.14}²⁺Mg_{0.26})^{-0.40}(Si_{3.93}Al_{0.07})^{-0.07}O₁₀(OH)₂]^{-0.47}[Ca_{0.21}Na_{0.05}K_{0.01} |
| montmorillonite | |
| Kinney | [(Al_{1.48}Fe_{0.06}³⁺Mg_{0.46})^{-0.46}(Si_{3.93}Al_{0.07})^{-0.07}O₁₀(OH)₂]^{-0.53}[Na_{0.50}]^{+0.50} |
| montmorillonite | |

TABLE 4
Comparison between bacterial viability and oxidation-reduction
potential (ORP) for various bacteria, for a variety of natural
clays, and for pyrite-amended clays. Suspensions were 40 mg
clay/mL water, unless otherwise noted, and experiments lasted
for 24 h.

| FIG. | | | Viable cells | ORP |
|---|---|---|---|---|
| Ref. | Bacteria tested | Mineral used | (% of total) | (mV) |
| 2A | Streptococcus | Culture control | 73.0 | 167.0 |
| | sp. | Argiletz | 84.9 | 81.1 |
| | | Wyoming | 57.2 | 98.6 |
| | | Argicur (heated 200 C.) | 16.6 | −96.2 |
| | | Argicur | 2.5 | −101.0 |
| | | Pyroclay | 1.2 | −48.1 |
| 2B | Staphylococcus | Culture control | 93.0 | 126.0 |
| | epidermidis | Miraculite | 99.8 | 144.0 |
| | | Pyrite only (1 h grind) | 7.4 | −49.0 |
| | | Pyrite only (0.5 h grind) | 1.1 | −32.0 |
| | | North Sea sediment | 0.17 | −91.0 |
| | | Pyroclay | 0.07 | −115.0 |
| | | Argicur | 0.03 | −122.0 |
| 2C | Escherichia | Culture control | 84.0 | 120.0 |
| | coli | Ormalite | 63.3 | 53.7 |
| | | Pyrite only | 3.5 | −58.0 |
| | | Pyroclay | 2.6 | −113.0 |
| | | North Sea sediment | 1.2 | −137.0 |
| | | Argicur | 0.5 | −175.0 |
| 3 | Escherichia | Pyroclay (0.4 mg/mL) | 44.8 | 68.0 |
| | coli | Pyroclay (4 mg/mL) | 28.7 | 62.0 |
| | | Pyroclay (20 mg/mL) | 33.7 | 55.0 |
| | | Pyroclay (40 mg/mL) | 0.2 to 2.6 | −113.0 |
| 5A | Staphylococcus | Wyoming with no pyrite | 44.5 | 2.1 |
| | epidermidis | Wyoming with pyrite | 3.4 | −35.1 |
| | | ground 0.5 h | | |
| | | Wyoming with pyrite | 1.6 | −55.4 |
| | | ground 1.0 h | | |
| 5B | Escherichia | Wyoming with no pyrite | 19.1 | −47.1 |
| | coli | Wyoming with 10% | 7.8 | −83.1 |
| | | pyrite ground 0.5 h | | |
| | | Wyoming with 10% | 2.8 | −56.7 |
| | | pyrite ground 1.0 h | | |
| 5C | Escherichia | Culture control | 95.8 | 167.0 |
| | coli | Kinney only | 60.6 | 2.3 |
| | | Kinney + 10% pyrite + | 31.7 | −78.4 |
| | | 10% calcite | | |
| | | Kinney + 1% pyrite | 22.6 | −86.4 |
| | | Kinney + 10% pyrite | 3.1 | −91.9 |
| 6 | Escherichia | Weathered Blue clay | 89.8 | 78.4 |
| | coli | Blue clay | 10.3 | −27.9 |

1. (canceled)

2. A synthetic bactericidal composition, comprising
a bactericidal effective amount of a particulate reducing agent selected from the group consisting of pyrite, marcasite, pyrrhotite, FeS₂, FeS, FeSO₄, and a combination thereof, and
a clay or clay mineral comprising a smectite clay, an illite clay, a rectorite clay, or a combination thereof.

3. (canceled)

4. The composition of claim 1, wherein said fine particulate reducing agent is present in said composition in an amount ranging from approximately 0.5% wt. to 10% wt. of said composition.

5. The composition of claim 2, wherein the particle size of said reducing agent is less than 1 micron.

6-7. (canceled)

8. The composition of claim 2, wherein said reducing agent is pyrite, and wherein said day is a smectite day.

9. The composition of claim 8, wherein said composition comprises 0.5% wt. to 10% wt. fine particulate pyrite; and wherein the particle size of said pyrite is less than one micron.

10. The composition of claim 8, wherein said composition comprises approximately 10% wt. pyrite and a Ca-saturated smectite clay.

11-18. (canceled)

19. A method of treating a bacterial skin infection, comprising topically applying to the site of said bacterial skin infection a composition as set forth in claim 2.

20. A method of treating a bacterial skin infection, comprising:
adding to a composition as set forth in claim 2 a suitable aqueous liquid in an amount so as to create a paste; and
topically applying said hydrated composition to the site of said bacterial skin infection.

21. The method of claim 20, wherein said suitable aqueous liquid is water.

22. The method of claim 20, wherein said bacterial skin infection is caused by one or more bacteria selected from the group consisting of Mycobacterium ulcerans, E. coli, ESBL E. coli, Pseudomonas aeruginosa, Salmonella typhimurium, Staphylococcus epidermidis, S. aureus, MRSA, M. smegmatis, Streptococcus sp. and M. marinum.

23. A method of making a synthetic bactericidal composition comprising:
adding a bactericidal effective amount of a reducing agent in fine particulate form to a non-bactericidal clay or non-bactericidal day mineral, wherein said reducing agent is pyrite, marcasite, pyrrhotite, FeS₂, FeSO₄ FeS; and
rendering said composition bactericidal.

24. (canceled)

25. The method of claim 23, wherein said fine particulate reducing agent is added to said clay or day mineral so as to be present in said bactericidal composition in an amount ranging from 0.5% wt. to 10% wt.

26. The method of claim 23, wherein said non-bactericidal day or said non-bactericidal day mineral are synthetic.

27. The method of claim 23, wherein said non-bactericidal day is a smectite day, an illite clay; or a rectorite clay.

28-33. (canceled)

34. A method of determining whether a day has bactericidal properties, wherein said method comprises chemically and/or mineralogically analyzing said clay to determine the presence of a reducing agent comprising pyrite or marcasite therein or measuring the oxidation-reduction potential of a slurry containing said day.

35-36. (canceled)

37. A synthetic antibacterial day or day mineral, wherein said antibacterial clay or day mineral is produced by:
synthesizing a clay or day mineral or by treating or altering the chemistry of a natural clay or day mineral to yield a synthetic antibacterial day or day mineral containing within its crystal structure or within its exchange positions an antibacterial effective amount of a reducing agent; wherein said reducing agent ferrous iron and renders said clay or day mineral antibacterial.

38-43. (canceled)
